(19) United States
(12) Patent Application Publication
(10) Pub. No.: US 2024/0038332 A1
(43) Pub. Date: Feb. 1, 2024

(54) SYSTEM AND METHOD FOR IMPROVED CARBON SEQUESTRATION BY UTILIZING IMPROVED GENETIC MODIFICATION OF ALGAE

(71) Applicant: John McDevitt, Clearwater, FL (US)

(72) Inventor: John McDevitt, Clearwater, FL (US)

(21) Appl. No.: 18/483,512

(22) Filed: Oct. 9, 2023

Related U.S. Application Data
(63) Continuation of application No. PCT/US22/25153, filed on Apr. 17, 2022.
(60) Provisional application No. 63/182,891, filed on May 1, 2021.
(60) Provisional application No. 63/235,703, filed on Aug. 21, 2021.
(60) Provisional application No. 63/215,495, filed on Jun. 27, 2021.

(51) Int. Cl. G16B 20/50; G16B 50/30

(52) U.S. Cl. CPC G16B 20/50; G16B 50/30

(57) ABSTRACT

A system and method that provides a process of genetically modifying algae or seaweeds in order to have their life cycle processes changed such that they grow in such a way that after a period of time they arrive at negative buoyancy causing the algae or seaweed to sink to the bottom of a body of water. The purpose of this is to cause the carbon or other elements in the algae or seaweed to be captured and deposited on the floor of the body of water where the carbon or other elements of the dead algae or seaweed is sequestered and are not released into the body of water nor the atmosphere for a long period of time. Furthermore, the genetic modification may be done in such a way that the targeted genetic characteristics are optimized and may not be passed to subsequent generations of the algae or seaweed. This process may occur in a computer virtual test environment, a controlled laboratory environment, or the natural environment.

### CROSS REFERENCE TO RELATED APPLICATIONS

This application claims priority to U.S. Provisional Patent Applications No. 63/182,891 filed May 1, 2021, No. 63/215,495 filed Jun. 27, 2021, No. 63/235,703 filed Aug. 21, 2021, and PCT International Filing No. PCT/US2022/025153 filed Apr. 17, 2022, the contents of which are incorporated by reference herein.

### BACKGROUND OF THE INVENTION

Over the course of hundreds of millions of years sea plants have grown, died, sunk to the bottom of the ocean, and became the fossil fuels that are now burned for energy. A great deal of the carbon contained in these hundreds of millions of years of growth and death processes have been released through the burning of fossil fuels in the last hundred years. It is imperative that an improved system and method of capturing and containing carbon is implemented that acts much more quickly than the original life cycle of growing and sinking sea plants in order to remove carbon from the atmosphere (or ocean) and sequester it from the rest of the environment.

Growing plants is one of the most efficient methods of utilizing atmospheric carbon dioxide through photosynthesis and capturing it in the cellular structure of the plants. However, if the plant is subsequently burnt or rots (decomposing) the carbon is released back into the environment. Thus, it is critical to place the plant material somewhere that it will neither burn nor decompose (retaining the carbon within the plant's structure) for a lengthy period of time. A few characteristics of such an environment that prevents the short-term release of carbon from plant material is that it is anaerobic (low oxygen), free of sunlight (unable to support photosynthesis), cold (slowing organic decomposition), and readily reached (not requiring a significant input of energy to place the plant material into this environment). The bottom of the ocean (and some deep lakes) in deep water (e.g., the abyssal plain, approximately 3,000+ meters deep) has all of these characteristics, however there is the need for a way to efficiently deliver millions or billions of tons of plant material into this region much more quickly than the hundreds of millions of years that it took to get the plant material there originally.

The terms seaweed, plants, aquatic plants, or algae are used generically (and interchangeably) these could mean any set (individual or in combination) of plant, plant like organism, algae, macroalgae, microalgae, protist (e.g., seaweed, red seaweed (or algae), brown seaweed (or algae), green seaweed (or algae), golden seaweed (or algae), other algae, autotrophs, etc.), or in alternative embodiments fungi, bacteria, heterotrophs, etc. that may live in or on water (fresh water, brackish water, salt water, or a combination of these). Specific types of seaweeds or algae are some of the best candidate types of organism for genetic modification, genetic engineering, genetic manipulation, or genetic editing (any and all of which are used as synonyms and may be referred to herein as genetic editing or GE). As an example, but not a limitation, of the invention described herein, GE to current common species of seaweed or algae is needed to achieve the described invention goal(s). A wide variety of approaches to genetic modification, especially for subsequent genotypes, phenotypes, exhibitions, manifestations, activations, or suppression of certain characteristics in the current or future generations, have been utilized for centuries. The complexity and specificity of genetic modification approaches have increased over time. Methods of GE include, but are not limited to, (these individually or in combination may also be referred to as GE); Selective Breading, Mutagenesis, Polyploidy, Protoplast Fusion, Transgenesis, and Genome Editing and have provided ways of genome manipulation that have resulted in genetic structures which met the goal(s) of the genetic modification or editing process(es). The role of specific sections of some organisms' DNA and RNA have become understood, with some specific genetic base pairs known to control given traits.

Recent developments in genetic editing (these individually or in combination may also be referred to as GE) such as clustered regularly interspaced short palindromic repeats (CRISPR) and especially CRISPR associated protein 9 (CRISPR/Cas9 or CRISPR-Cas9), CRISPR Prime and other CRISPR related approaches including but not limited to; Cas3, Cas5, Cas8a, Cas8b, Cas8c, Cas9, Cas10, Cas10d, Cas12, Cas12a, Cas12b, SpRY Variant Cas12b, Cas12c, Cas12d, Cas12e, Cas12f, Cas12g, Cas12h, Cas12i, Cas12k, Cas13, Cas13a, Cas13b, Cas13c, Cas13d, CasX, Cmr5, Cse1, Cse2, Csf1, Csm2, Csn2, Csn4, Csx10, Csx11, Csy1, Csy2, Csy3, GSU0054, C2c4, C2c8, C2c9, CRISPR-Act3.0, SuperFi-CAS9, or other analogous approaches (collectively referred to as CRISPR herein, which also are included in GE) have been utilized to adjust (including but not limited to, gene activation or disabling, or changed functionality for one or more genes) the entire genome or any subset of the genome (and related phenotype or genotype) of a given set of genetic content or organism and potentially the progeny (by way of sexual or asexual reproduction) of the genetically edited genetic content or organism. Additionally, another approach that is included in this described system and method as GE is Base Editing—a CRISPR-Cas9-based genome editing technology that allows the introduction of point mutations in the DNA without generating DNA double strand breaks (DSBs). Two major classes of base editors have been developed: cytidine base editors or CBEs allowing C>T conversions and adenine base editors or ABEs allowing A>G conversions. Base editing is a novel technology that has the potential to generate gene knockouts or to correct certain errors or mutations in the DNA of intact cells. Another related GE approach is Prime Editing. It should be noted that under many definitions a genetic knockout (the removal of one or more genes) does not result in a genetically modified organism (GMO)—but is still considered as part of GE. While knockouts provide permanent changes, the system and method described may also include genetic knockdowns which provide for temporary changes (also considered to be part of GE). Other approaches to GE include, but are not limited to, knock-ins, homologous recombination, site-specific nucleases, zinc finger nucleases (ZFNs), transcription activator-like effector nucleases (TALENs), Retron Library Recombineering (RLR) Platform, Cas-CLOVER Nucleases, Mini-Cas9 Enzymes, NgAgo Protein Gene Editing System, CRISPR-Cpf1 System, FANA Antisense Oligonucleotide (FANA ASO) technology, meganucleases genetic editing, selection, mutagenesis, conjugation, and protoplast fusion. These genetic modifications are generally for the purpose of maximizing or minimizing (exhibiting or suppressing) a particular trait or set of traits (or characteristic(s)) for some benefit, however, given the very high complexities and intricacy of genetics in general (e.g., interdependent systems), and the added complexities and intricacy of the external environment (e.g., multiple interconnected interdependent systems) in which the organism exists, final results of the modification in the genetic code or the subsequent generations of the genetic code may no longer have the desired trait (or characteristic(s)) or the originally desired trait (or characteristic(s)) may actually be found to be detrimental to the genetic code, organism, species, or the broader environment. Furthermore, there is the risk of other unintended results stemming from the modifications, and thus requires a highly skilled approach to ensure successful achievement of goals.

While these GE tools are extremely powerful, the changes they can make are introduced very quickly and at specific locations in the genetic sequence at the direction of the user, not the result of evolution or adaptation over time. Given the complexity of the internal genetic systems and the compounding complexity of the external environment, the risk of failing to achieve the goal(s) of the genetic editing is extremely high. Furthermore, the editing may result in unintended genetic consequences that may create expensive mistakes—possibly even introducing detrimental genetic structures into the broader environment. Thus, the genetic modification may be done in a way such that any genetic change is just to the individual organism and is not transferable (by sexual or asexual processes) to later generations, or other species (through cross breeding). The described invention provides a more robust solution that is required to ensure long term capture and containment (sequestration) of carbon by way of genetically modified plant material.

### SUMMARY OF THE INVENTION

Accordingly, there is a need for a method and system that supports modifying the genetic content of seaweed or algae such that it grows (via photosynthesis), loses buoyancy at a point in its lifecycle, and sinks to the bottom of a body water in order to capture and sequester carbon. This sinking may be, but is not limited to, a loss of buoyancy of the algae such that it sinks in the deep-water ocean (e.g., to the abyssal plain) or other body of water. Additionally, the described system and method may also be constructed such that it does not introduce unintended or detrimental modified organisms into the environment. As an example, but not limitation, a general embodiment of the system and method may be described in summary as follows: A target species of seaweed is identified, the genetic structure of individual seaweed is altered such that the pneumatocysts (the gas filled sacks that allow some seaweeds to float on or near the water surface—also referred to as air bladders or floats) fail to maintain the seaweed's positive buoyancy at a certain point in its lifecycle. Seaweed with the modified (edited) genetic structure is placed or grows on the surface of the ocean where the seaweed continues to grow until the pneumatocysts fail keep the seaweed buoyant and the seaweed sinks to the bottom of the ocean where it is deep enough, anaerobic enough, dark enough, and cold enough that the seaweed decomposes minimally at a very slow rate—causing the cellular structure of the seaweed to remain mostly intact (including the carbon) on the ocean bottom, not re-entering the carbon/carbon dioxide cycle, for a long period of time (possibly over 1,000 years). In general, the described system and method enables the capturing and sequestering of carbon (generally in the form of carbon dioxide via photosynthesis) from the atmosphere and the ocean in the structure of the growing seaweed such that the sequester of carbon exceeds the release of carbon (in the form of carbon, carbon dioxide, methane, carbon monoxide, or other similar carbon containing molecules that may dissolve in water or escape into the atmosphere) from the decomposition of the dead seaweed. The carbon output required (including the energy that is the product of the use of fossil fuels to power systems) to grow and deposit the seaweed on the bottom of the ocean is less than the amount of carbon sequestered in the sunk seaweed—providing a net positive carbon capture amount. Other elements, individually or in the form of compounds and molecules that are part of the seaweed, including but not limited to, nitrogen, nitrates, methane, phosphates, and phosphorous, may be similarly sequestered. The sequestered carbon (or other elements) may be in a variety of forms including but not limited to the seaweed structure, particulate carbon (or other elements), and recalcitrant dissolved organic carbon (or other elements). Furthermore, the described system and method may be done from a small up to a very large scale—efficiently capturing and sequestering massive quantities of carbon (or other specific elements).

The exemplary system and method identify and structures modifications of genetic material in aquatic plants to achieve a given set of goal(s). Generally, goal(s) are the maximizing or minimizing a particular trait or set of traits for some benefit. This type of solution would save a great deal of time and resources in carbon capture and sequestering—resulting in materially improved results. Given the very nature of genetic editing, timely understanding of the results of any given edit is critical—saving even a portion of the time that makes up the lifecycle of the genetic material (genetic content) or of an entire organism would result in a minimum of an arithmetic savings of time and if multiple simultaneous (or slightly offset in time) edits are occurring the time savings could be exponential. While time is often the most critical element in such matters, there would also be material savings in resources (users (scientists), facilities, biological resources, machines, etc.), and also potentially the broader ecosystem. Each of these savings individually or in combination would result in significant expense savings. Additionally, a timely and efficient solution would be a benefit to the environment as a whole while reducing environmental carbon, the related global warming, and ocean acidification.

Accordingly, there is a need in the industry for a method and system that improves the results of genetic editing of aquatic plants (or plants or other organisms that are genetically modified or created to live in or on an aquatic environment) to support beneficial carbon capture and sequestration (generally long term). The system and method disclosed herein provides a rule based genetic editing system. The term genetic editing (“Genetic Editing” or “GE”, “GEs”, “GEing”) as used herein may include, but is not limited to, the manipulation (the adding, removing, re-positioning, changing, altering modifying, moving, substituting, creating by any process, including but not limited to selective breeding, CRISPR, base editing, prime editing, knockouts, knockdowns, or other similar processes) any portion of genetic content (GC) including the gene bases—adenine, cytosine, guanine, and thymine (“A”, “C”, “G”, and “T”) (or their analogues, including but not limited to uracil in RNA), genes, or alleles, individually, in groups (including RNA, DNA, and any analogous material or structure—including single or double strand DNA), up to and including the entire genome. These GEs may be temporary or permanent in a given organism, clones of an organism (clones generally meaning organisms created by way of asexual reproduction or replications including, but not limited to, budding, fracturing/fragmentation, binary fission, vegetative reproduction, spores, parthenogenesis, regeneration, or similar replication of an organism or a group of organisms), or later generations of the organism (or cross-bred later generations) where the resulting traits (or characteristic(s)) may be visible, invisible, recessive, dominant, exhibited in the genotype, or phenotype of the organism, clones of the organism, or resulting generations (of the genetic material or the organism). Additionally, these GEs may be done to DNA, RNA, or any analogous genetic structure (or portion of genetic structure) in whole or in part. These GEs may occur completely once (in a single or in multiple organisms, to all the genetic material in an organism, an individual piece of genetic material, or a set/subset of genetic material), or in stages over time (in one piece of genetic material, a set/subset of genetic material, or all of an organism's genetic material), or at once or in stages over time in multiple organisms (in one piece of genetic material, a set/subset of genetic material, all of an organism's genetic material) and this could include over multiple generations of genetic material in a given organism, multiple organism clones, multiple generations of an organism, or a population of organisms. The GEing process may be done in parallel, in sequence or in combination across any of the aforementioned groupings.

All the genetic material that is capable of being involved in GE (directly or indirectly) (physically, digitally, or in combination) may be referred to as Genetic Content (or “GC”). Additionally, the GC may be actual physical material, digital virtual material, or a combination of the two. Also similarly, the GE may be done in the physical world (including a controlled laboratory environment, or an uncontrolled natural environment), the digital virtual world, or a combination of the two. The GE structure may first be arrived at and analyzed in a digital environment (possibly using AI/ML) where the GC information is edited and then the GE that is identified as successful in the digital environment is performed in the physical environment—creating edited GC that is coordinated and congruent with the structure of the edited genetic content information. The mechanisms utilized to do the GE to the GC will be described in greater detail below. However, in the exemplary embodiment the GE to GC may be done in such a way to ensure that the modification is not inheritable to later generations and in some cases the GC may be sterile—incapable of reproduction by any process, by sexual reproduction, asexual reproduction, or cross reproduction. Additionally, specific individual processes of reproduction e.g., sexual or asexual (including but not limited to binary fission, budding, vegetative reproduction, fragmentation, or spores) may be blocked from occurring. In alternative embodiments reproduction (sexual or asexual) may occur, but only under specific designated conditions. It should be noted that the primary goal of the GE in the example case is to have the given algae grow, lose buoyancy, sink, and take the captured carbon to the bottom of the ocean causing the captured carbon to be sequestered for a lengthy period of time. This sinking provides a degree of safety from the risk of unintended over-growth (or bloom) of the genetically edited algae. Thus, even if the genetically edited algae escape from the test environment, it will still sink in the bottom, preventing over-growth. The possible addition of genetic modification to cause the genetically edited algae to be sterile provides even more assurance that an unintended or accidental introduction of the genetically modified algae to an ecosystem will not have a detrimental impact on the ecosystem. Instead of a population explosion—the genetically modified algae would just be present for a limited period on the surface and then sink away—unable to reproduce. Furthermore, the GE may be performed on one or more types of GC that may act alone or in coordination with each other to achieve the goal(s).

The system and method disclosed herein has a variety of rule sets but collectively these rule sets are used in a coordinated way, taking into consideration goals and constraints, to arrive at desired final outcomes manifested in how GE to GC meets the goal(s), and these goal(s) may not just be individual genetic structure, or organism performance, but how that genetic structure meets the goal(s) of the broader ecosystem in which it exists. Please note the term “sets” is used generically herein and may mean any collection of a given thing (including the null set (an empty set), a singular set (a set with a single component), or multiple set (a set with multiple components, including subsets)). This system and method is not limited only to the specific gene bases, but rather also, the broader impact of the GE to sets of gene bases (including those that may not have been specifically edited, or those that do not naturally occur). Furthermore, the system and method is not just addressing one organism's strand(s) of GC (RNA or DNA or analogous structures), but rather the complex interaction and possible unintended consequences of GE to one or more than one set of GC in one or more than one organism (e.g., modifications may include changes to RNA or DNA in one organism, as well as, changes to multiple organisms—that could be genetically related or unrelated organisms in an ecosystem—thus there may be multiple GEs to multiple GC simultaneously or over time).

The term rule(s) is used generically (often in the simplest form being If-Then statements) and may include, amongst other things, set(s) of rules including, amongst others, ecosystem rules around processes and results (e.g., inclusions, exclusions, optimization, maximization, minimization, commercial rules, business rules, legal rules, ethical rules, etc.), genetic content rules (e.g., inclusions, exclusions, complexities, similarities to other organisms, etc.), predictive rules (e.g., math rules, probability rules, statistical rules, modeling rules, quantitative rules, qualitative rules, etc.), genetic structure rules (e.g., biological rules, structural rules, bio-chemical rules, physical rules/limitations, etc.), and randomization rules. All of these rules may be individualized or grouped, they may be preferences, relative preferences, relative requirements, or absolute requirements, they may be interdependent partially or wholly, they may be hierarchical in nature, they may be sequential, they may occur in parallel, or a combination of any of these. Furthermore, these rules may function as logical engines that may organize, prioritize, include, exclude, change the likelihood, etc. of a given individual GE (or set of a GEs) to be used. The rules may be set by an individual, group, a system, a computer, another set of rules, hierarchical rule sets, or a combination of any of these. The rules may be pre-established, dynamically established, established in phases, or a combination of any of these. The results of the GEs may be evaluated by a user of the system. Please note the terms; “results”, “product”, “outcome”, may be used interchangeably, generically, and could mean the results (the exhibited, hidden, suppressed, dominant, recessive, latent, visible, invisible, temporary, permanent, multigenerational, or inter-organism manifestations in the organism, multi-organism, the entire ecosystem, or any subset). Similarly, “reviewer”, “user”, “viewer”, “evaluator”, and “consumer” are used interchangeably, generically, and could mean any reviewer of the results of the GEs and the user could be a human individual, a group of humans, an organism or organisms, a computer system, or set of systems. Additionally, the terms “review” or “evaluate” are used generically, interchangeably, and can mean any method of consumption, review, analysis, etc. by a user of the GE results.

The term “sink” is used generically meaning going lower in water and may be the product of any loss of buoyancy such that the seaweed becomes denser than the surrounding water and descends. This sinking may result from a variety of pneumatocyst failures, including but not limited to (individually or in combination), pneumatocyst integrity failure (including but not limited to any one or combination of, pneumatocyst breaking, leaking, shrinking, etc.), insufficient pneumatocyst growth (including but not limited to any one or combination of, insufficient pneumatocyst size, number, gas mixture, gas retention, gas density, etc.), non-pneumatocyst seaweed over-growth (including but not limited to any one or combination of, higher rate of growth of non-pneumatocyst plant portions, higher accumulation of heavier elements, more dense cellular structure, etc.), or other similar cause of loss of buoyancy of the seaweed. Furthermore, the sinking of the seaweed may be caused (individually or in combination with other factors) by the addition of a secondary organism (which may be one or more organisms (or parts of organisms) which may or may not be the subject of GE) to the host (or primary) seaweed that may grow in, on, or with the host seaweed such that it causes the host seaweed to lose buoyancy and sink. The primary or the secondary GC may either, neither or both be the subject of GE. As an example, but not limitation, the secondary organism may be a mollusk or coral that through biomineralization creates structures from calcium carbonate or other substances (further increasing the carbon sequestration (greater amount of carbon—stored for a longer term) or density of the combined seaweed with secondary organism). Additionally, the sinking of the seaweed may create a current that causes an upwelling of nutrients required (including but not limited to iron, nitrogen, nitrates, phosphates, etc.) to help support additional growth of seaweed. By way of example, but not limitation, seaweeds or algae that float at or near the surface of the water or have some form of pneumatocyst include sargassum, kelp, fucus vesiculosus (e.g., bladder wrack), etc.

In general terms the example system and method described herein, effectuates the sinking of seaweed by way of GE seaweed GC (or alternatively the addition of a secondary organism GC, or the GE of secondary GC). Furthermore, it should be understood that the example system is not a limitation on the described system and method which may in addition to effectuate the sinking of seaweed, may also effectuate the timing or point in lifecycle that the seaweed sinks, the rate of growth of seaweed, the carbon (or other elements) capture rate, the ability to sequester carbon, the nutrient requirements for growth requirements (including but not limited to iron, nitrogen, nitrates, phosphates, etc.), the growth volume to captured carbon (or other elements) ratio (the density of capture), the size seaweed (or parts of seaweed) grows to, the rate or size different portion of a seaweed plant grows (for example but not limitation the pneumatocysts grow more slowly than other parts of the seaweed in a portion of the growth cycle), the seaweed growth area albedo, the rate of growth of the secondary organism, the rate of carbon capture of the seaweed (or secondary organism), the total amount of carbon captured by the seaweed (or secondary organism), or other elements, molecules, or compounds (including but not limited to oxygen, nitrogen, phosphorous, metals, metalloids, nonmetals, carbonates, phosphates, nitrates, etc.) captured by the seaweed (or secondary organism), the ability for the seaweed plant to remain structurally together, the ability of the seaweed to sink as a single unit, the cellular integrity of the seaweed such that it stays whole for a longer period of time, the duration of the sequestration of carbon (or other elements), the limitations on reproduction of the seaweed, restrictions on the ability of the seaweed to reproduce, the ease and accuracy of monitoring, measuring, and reporting on seaweed growth, the location of the sinking, the time of sinking, the rate of sinking, the mass of elements that reach depth, the rate of decay, the amount of elements that remain sequestered, the length of sequestration, rate of decomposition, the timing of the sinking of the seaweed (or secondary organism), the mass of elements captured per area per time period, sterility/ability to reproduce/replicate, ability to cross-pollinate, overall impact on the environment, unintended consequences, etc. Also, it should be noted that in alternate embodiments of the described system and method the GE of the GC may be performed to effectuate a seaweed structure or a rate of sinking that enables the GE seaweed (or secondary organism) to pull down or sink other materials in the water—including but not limited to other seaweeds, plastics, micro plastics, oils, trash, detritus, flotsam, etc. that are floating or are suspended at or near the surface of the ocean (or in the path of the sinking seaweed), such that it would be beneficial to have this content sequestered in deep ocean locations. Furthermore, in other alternate embodiments of the described system the GE of the GC may be done to increase the growth rate of the seaweed, increase the growth rate of secondary organisms, reduce the lifecycle of the seaweed, increase the capture and sequestering of various elements, molecules, and compounds (including but not limited to, carbon, nitrogen, phosphorus, phosphate, nitrates, etc.). Additionally, the genetically modified seaweed may be used the take-up excess nutrients in water, where the genetically modified seaweed may use the nutrients in the water, sequester the component elements in the cell structure, and then sink, with the excess nutrients sequestered in the cell structure. Thus, preventing an over-growth or bloom of other algae (potentially detrimental over-growths such as the sargassum blooms in the Atlantic) due to the excess nutrients.

A set of rules are applied to starting genetic material, which may include among other things a set of gene bases (A, C, G, & T or their analogues, including but not limited to uracil in RNA), (partial or complete with original or substituted organic material), a full or partial piece of GC, an organism, reproductive elements of an organism, or a collection of organisms. Furthermore, genetic material may be removed, added, re-positioned, supplemented, created, altered, or moved (individually or in combination) in relation to the starting genetic material by way of GEing tools such as selective breeding, CRISPR or other GE methods following standard and emerging industry processes.

The GE may also include changes to reduce the likelihood that the genetically edited seaweed is sexually reproducible (or cross-breed-able). This may be achieved by processes such as selective breeding, CRISPR, or other GE methods. These methods may prevent or reduce the meiosis stage of development such that sexual reproduction or cross breeding is not possible and only asexual (or clone like) reproduction is possible. Furthermore, the GE may include changes to reduce the ability for the genetically edited seaweed to asexually reproduce, including but not limited to, any or all of the main types of asexual reproduction; binary fission, budding, vegetative reproduction, fragmentation/fracturing, spores, parthenogenesis, regeneration, or any similar method. Alternatively, GE may also include changes to increase the likelihood of sexual or asexual reproduction of the seaweed. Furthermore, the GE may be done to only allow one type of reproduction. Additionally, any secondary organism that is added to the primary seaweed may also be GE such that its likelihood of reproduction is changed similar to the ways in which the primary seaweed is changed.

The results of the final genetic structure of the GC—including any or all of the genome itself, the organism, clones of the organism, the progeny of the organism, groups of organisms, secondary organisms, and the ecosystem—are reviewed and analyzed to evaluate how the final results compare to the initial goal(s). These evaluations may occur at one or more points in time (taking into consideration delayed impacts) to measure robustness and success of the GE. It should be noted that this system and method disclosed herein has an inherent feedback loop and the entire process or any subset(s) of the process may happen once or multiple times and may occur in an iterative way where the same GC, organism, clones, related organisms (primary & secondary organisms), or groups of organisms may be edited multiple times (in parallel or in series) over time where the results may be reviewed and then depending on the nature of the results additional cycles through the system may occur. These processes may occur once or multiple times in rapid or delayed succession (also in parallel, in series, or a combination of these).

Alternatively, the described system and method may also achieve the goals by introducing a secondary organism(s) (which may include, but is not limited to, one or more of microbes, mollusks, animals, plants, bacteria, protozoa, archaea, algae, other seaweed, etc.) to the primary (host) seaweed that is to be sunk. Both the secondary organism or the host seaweed may have GE to their GC. The GE may be achieved by processes of selective breeding, CRISPR, or other GE processes. The secondary organism may act in a coordinated way with the primary or host GC and have a symbiotic, parasitic, mutualistic, commensalistic, or competitive relationship with the host seaweed. The secondary organism may grow in, on, or with the host seaweed in such a way that supports the sinking of at least the primary seaweed. Additionally, the secondary organism may act in such a way to optimize the growth rate, the life cycle duration, the carbon sequestration (and term of storage), the other element sequestration (and term of storage), or other performance of the host seaweed (or host seaweed and secondary organism(s)) such that the goals are achieved. Furthermore, a possible benefit of the introduction of a secondary organism to the host organism where there may be GE to the GC, may be the reduction of the risk of introducing a sexually, asexually reproducing or cross breed-able organism into the environment—it may be the case that the secondary organism (which does not naturally occur with the primary seaweed) is required for the primary organism to achieve its goal.

All of the processes of the disclosed system and method may occur in the physical world or virtually in a digital environment or a combination of the two. Furthermore, the disclosed system and method may be structured of at least one (or more) database, and at least one (or more) processor (may be described as a server) configured to be communicatively coupled and configured to act in a coordinated way. By example but not limitation, the at least one genetic content database may store actual physical organic material, or alternatively it may store information data that describes organic material, or it may store both physical and digital material. The at least one (or more) database and the at least one (or more) processor may be physically located together or apart (e.g., in the cloud). The at least one processor(s) have resident software (and additional software may be added over time) to enable the at least one processor(s) to perform the disclosed activities including but not limited to acting as rules engines. The described system and method may utilize computing tools for many parts of the process (especially the make-up of the GC, the modeling of the potential genetic structure, the finalized genetic structure, and the evaluation of the results of the GE, the impact of it, and the iterative feedback process) these computing tools may be traditional classical computing, quantum computing, momentum computing, or a combination of these. These computing processes may also use artificial intelligence/machine learning (AI/ML) systematic approaches to increase the efficiency and performance of the system, improving quality while reducing time and other system resources. These processes may occur in series, in parallel, or both.

The system disclosed herein is a system for selecting and genetically editing existing GC in order to achieve a set of goals of carbon capture and sequestration (generally for a long term) while minimizing resource consumption, the system comprises: at least one electronic database configured to store at least one set of information related to existing GC configured to be genetically edited by way of the addition of information related to at least one set of new GC stored in at least one electronic database, the removal of at least one set of information related to existing GC the identity (or descriptive information) of which stored in at least one electronic database, or the moving or re-positioning of at least one set of information related to existing GC, the identity (or descriptive information) of which is stored in at least one electronic database; at least one processor with software instructions stored therein that, when executed by the at least one processor, configure the at least one processor to execute: a base genetic structure rules engine configured to generate at least one approved genetic structure characteristic based on at least one ecosystem rule; including a predictive rules engine configured to generate at least one GC structure characteristic based on at least one historical GE result set. Also, a base genetic structure engine configured to generate a set of genetically edited GC by at least one of; genetically editing at least one set of GC, adding at least one set of new GC, moving or re-positioning at least one set of information related to existing GC, removing at least one set of existing GC—to or from the at least one set of existing GC such that the process resource consumption is minimized by creating genetically edited GC that is most likely in compliance with the at least one ecosystem rule and goal. Furthermore, at least one processor configured to enable the genetically edited GC to exhibit the results of the genetic editing, wherein the results of the genetic editing are evaluated as to their relative achievement of the at least one ecosystem rule and goal, wherein at least one predictive rules engine is further configured to: receive updates (periodically or continuously based on pull or push commands) related to at least one other historical result and at least one genetically edited structure result, and correlate the at least one result of the GE with the at least one exhibited characteristic and compare the at least one result with the at least one ecosystem rule and goal. Furthermore, it should be noted that the system elements are performed in a coordinated manner and may each be performed in part or in whole and in any logical order with actual physical GC, information that describes GC, or a combination of these. The described edit of GC may start with genetically editing the information that describes the genetic content (this may be done using AI/ML) such that genetic content structures that are in compliance with goals and rules (at the organism, family, species, or ecosystem level) are able to be identified. Once the required genetic content structure is identified, the genetic content may be GE such that the GC is in coordination and congruent with the genetic information structure. Additionally, following the start of the process the system elements may occur automatically continuously or discretely with or without user involvement, or may require user involvement for each phase of the process (or a combination of both).

Furthermore, the disclosed system includes a genetic structure rule set engine, which may be further configured to calculate a set of approved genetic structure(s) based on at least one other historical result of GEing. The disclosed system also has at least one predictive rules engine that is configured to calculate at least one “best guess” genetic structure based on at least one set of evaluated results and may be configured to calculate at least a second “best guess” GC structure characteristic based on at least a second set of evaluated results. Furthermore, the disclosed system has at least one predictive rules engine that is further configured to receive evaluated results data related to at least a first GC GE and calculate at least a second “best guess” genetic structure characteristic in response to a more than first GC GE. Also, the disclosed system has at least one genetic structure rule set engine that is configured to calculate a weighting for the selected and randomized set(s) or subset(s) of the plurality of possible genetic structures, and the probability of at least one finalized genetic structure is created in part based on the calculated weighting. Computing tools including AI/ML may also be utilized to arrive at the potential “best guess” initial solutions. Furthermore, the algae to be genetically edited may be sargassum and other goals for the GE process may include for example, but not limitation; increasing the ability and amount of carbon captured (e.g., more carbon per algae volume per time period), increasing the accuracy of the timing of the sinking of the algae (increasing or decreasing the time until the algae reaches the sinking point in its life cycle), restricting the ability of the algae to reproduce, increasing the structural integrity of the algae, or increasing the period of time the carbon is sequestered.

The method disclosed herein for selecting and genetically editing existing GC in order to achieve a set of goals related to carbon capture and sequestration while minimizing resource consumption includes; storing in at least one electronic database configured to store at least one set information related to existing GC configured to be genetically edited by way of the addition of at least one set of information related to at least one set of new GC stored in at least one electronic database, or the removal of at least one set of information related to existing GC the identity (or descriptive information) of which stored in at least one electronic database, or moving or re-positioning at least one set of information related to existing GC the identity (or descriptive information) of which is stored in at least one electronic database. Also, generating by at least one processor with software instructions stored therein that, when executed by the at least one processor, configure the at least one processor to execute: generating by at least one base genetic structure rules engine configured to generate at least one approved genetic structure characteristic based on at least one ecosystem rule; applying a predictive rules engine configured to generate at least one GC structure characteristic based on at least one other historical GEing result set; and applying a base genetic structure engine configured to generate a set of genetically edited GC. This may be achieved by at least one of; adding at least one set of new GC or removing at least one set of existing GC to or from the at least one set of existing GC, moving or re-positioning at least one set of information related to existing GC, such that the process resource consumption is minimized by creating genetically edited GC that is most likely in compliance with the at least one ecosystem desired outcome (rule and goal). The disclosed method also may utilize at least one processor configured to enable the genetically edited GC to exhibit the results of the genetic editing, wherein the results of the genetic editing are evaluated as to their relative achievement of the at least one ecosystem rule and goal. Also, where at least one predictive rules engine is further configured to: receive updates (periodically or continuously based on pull or push commands) related to at least one other historical result set and at least one genetically edited genetic structure result and correlate the at least one result of the GE with the at least one exhibited characteristic and compare the at least one result with the at least one ecosystem rule and goal. Furthermore, it should be noted that the method elements are performed in a coordinated manner and may each be performed in part or in whole and in any logical order with actual physical GC, information that describes GC, or a combination of these. Additionally, following the start of the process the method elements may occur automatically continuously or discretely with or without user involvement, or may require user involvement for each phase of the process (or a combination of both).

The disclosed method includes the genetic structure rule set engine which may be configured to generate the set of approved genetic structure(s) based on at least one other historical result of GEing. Also, the disclosed method may include at least one predictive rule engine that may be configured to generate at least one “best guess” genetic structure based on at least one set of evaluated results and also configured to generate at least a second “best guess” GC structure characteristic based on at least a second set of evaluated results. Furthermore, the disclosed method may include at least one predictive rules engine configured to receive evaluated results data related to at least a first GC GE and may also generate at least a second “best guess” genetic structure characteristic in response to a more than first GC GE. Additionally, the disclosed method may include at least one genetic structure rule set engine that may be configured to determine a weighting for the selected and randomized subset(s) of the plurality of possible genetic structures, where the probability of at least one finalized genetic structure is created in part based on the determined weighting.

By way of example, but not limitation, an example implementation of the described method and system could be as follows: There is a desired goal in an ecosystem to reduce carbon dioxide in the atmosphere or dissolved in ocean water and to achieve this goal there is another goal of developing a floating algae that can utilize carbon dioxide via photosynthesis and capture the carbon in the algae cell structure. Additionally, there is the goal of genetically modifying the floating algae such that once the algae reaches a point in its growth cycle the pneumatocysts that keep the algae fail to maintain the algae's buoyancy and the algae sinks to the bottom of the ocean (at the bottom of deep water algae decays very slowly such that the carbon may be sequestered for 1,000+ years). Furthermore, there is the rule that the genetically modified algae does not cause any unintended harm to the larger ecosystem from unlimited growth of the genetically modified algae.

In this example case, sargassum may be selected to be the genetically modified algae to achieve the goals while adhering to the rules. The existing genetic make-up of the sargassum can be analyzed to identify the portion of the genetic structure that is related to the pneumatocysts. Computer modeling (including AI/ML approaches) may then be applied to identify the required modification of the sargassum pneumatocysts to cause the pneumatocysts to no longer be sufficient to maintain the buoyancy of the sargassum—such that it sinks to the bottom of the ocean. Different genetic editing techniques and genetic edits may be reviewed to arrive at a proposed process that provides a “best-guess” of genetic content that is the result of genetic editing that would achieve the goals and adhere to the rules that have been set. The genetic editing may be performed, a final set of genetic content is created, and the resulting genetically modified sargassum may be grown to see how it develops. The results of the genetically modified sargassum are evaluated and in this example case they may be found to work well, but not as well as would be liked and there may be concern that the modified sargassum may have negative impacts on the larger ecosystem. Thus, another cycle of genetic modification is needed (informed by the first process cycle). The process may then be repeated until a final edited genetic content that satisfies the goal(s) and meets the rule(s).

In this example, there is the desire to restrict the ability of the sargassum to reproduce and for genetically modified sargassum to be sterile such that it does not reproduce and over-grow, nor introduce a detrimental genetically modified algae into the ecosystem. By removing a portion of the genetic content structure, the modified sargassum may not be able to reproduce in the wild, nor may it be able to crossbreed with existing sargassum. Additionally, the genetic content related to the rate of pneumatocyst failure is further adjusted to ensure that the timing of the failure to maintain buoyancy for the entire plant occurs at approximately the same time such that the entire plant sinks all at once. This combination of sterility and buoyancy loss rate provides a kind of “circuit-breaker” such that if any of the genetically modified sargassum escapes out of the test environment it will just sink harmlessly to the bottom of the ocean and will not impact the rest of the ecosystem. In this example case, the latest genetically modified sargassum is grown and the results are evaluated. It is felt that the genetically modified sargassum could be further modified to increase the amount of carbon dioxide captured. The modification and evaluation process occurs yet again to check to see if the goals are achieved without violating the rules. Once there is successful goal achievement and rule adherence the genetically modified sargassum may be tested in a lab environment. If the testing is passed in the lab environment, the genetically modified sargassum is tested in large tanks. If the large tank testing is passed the genetically modified sargassum is tested in open water netted farms. If the testing is passed in the open water netted farms, the genetically modified sargassum is tested in un-bounded open water. If the un-bounded open water testing is passed, then the genetically modified sargassum is grown at large scale. However, if any stage test fails then the modification and evaluation process start again in an iterative process.

### BRIEF DESCRIPTION OF THE DRAWINGS

FIG. 1 illustrates a block diagram of a system for creating and evaluating modified genetic content in accordance with an exemplary embodiment.

FIG. 2 illustrates a flowchart for a method of creating and evaluating modified genetic content in accordance with an exemplary embodiment.

FIG. 3 illustrates an example of a general-purpose computer system in accordance with an exemplary embodiment.

### DETAILED DESCRIPTION

The following detailed description outlines possible embodiments of the proposed system and method disclosed herein for exemplary purposes. The system and method disclosed are in no way meant to be limited to any specific combination of hardware and software. As will be described below, the system and method disclosed herein relate to the GE of GC to achieve a set of goal(s) in accordance with a set of rule(s). It should be appreciated that each of the components in the figures below are illustrated as simple block diagrams, but include the requisite biological, mechanical, physical, digital, hardware, and software components needed to perform the specified functions as would be appreciated by one skilled in the art. For example, one or more of the components described below can include one or more databases, one or more computer processor units (CPUs) configured to execute software programs stored on electronic memory in order to execute the algorithms disclosed herein, these databases and CPUs may be located together or apart, physical, or virtual, and may be classical, quantum, momentum or a combination of these types of computer processors. In general, the term computer can refer to classical computing, quantum computing, momentum computing, artificial intelligence, machine learning, and any combination or subset of these, and these approaches may be applied sequentially or in parallel (or a combination of these) and amongst other things may function as rules engine in performing the various tasks herein. It should be noted that from the following discussion, alternative embodiments of the systems and methods disclosed herein will be readily recognized as viable alternatives that may be employed without departing from the principles of what is claimed.

Reference will be made in detail to multiple embodiments, examples of which are illustrated in the accompanying figures. It is noted that wherever practicable similar or like reference numbers may be used in the figures and may indicate similar or like functionality. The figures depict embodiments of the disclosed system and method for purposes of illustration only. One skilled in the art will readily recognize from the description that alternative embodiments of the systems and methods illustrated herein may be employed without departing from the principles described herein.

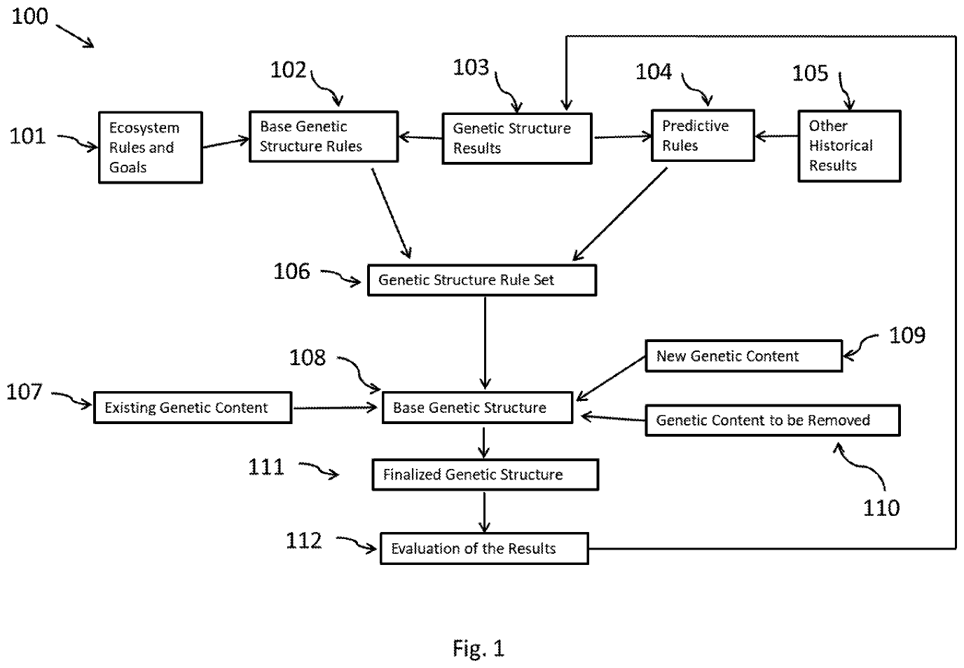

For example, but not limitation, FIG. 1 is a basic exemplary example of the genetic modification of GC to make a more robust set of GC that meets the goal(s) and adheres to the rule(s) of the described system and method. In this case the system can be considered a Genetic Content Modification System (100). This system (GCMS) is capable of acquiring GC, identifying GC, modifying GC, editing GC, combining GC, evaluating edited GC, and then depending on the results, revising, and re-processing the GC multiple times. The GC to undergo GE may be of the primary (host) organism or the secondary organism(s). The specific components and functionality of the GCMS will be described in more detail below. It should be noted that each of the following elements may be performed systematically and automatically with or without user intervention, or each may also be performed with a user override. Additionally, the rules of this system (100) may be pre-set or may be dynamically adapted in real-time/near real-time (continuously or periodically), and the adaptations may be based on the information that is available at that time, and also as additional information becomes available the rules may be further dynamically (continuously or periodically) adapted. Furthermore, the rules system (100) may be updated or adapted periodically or continuously based on pull or push commands internal or external to the system. These changes may be based on either or a combination of user/AI/ML input and will be described in more detail below. The GE may be performed by selective breeding, CRISPR, or other GE methods. Furthermore, the entire process may occur with multiple sets of GC and the phases may be nested with different sets of GC being processed at separate times, synchronously or asynchronously.

According to the exemplary example, the GCMS (100) has a set of Ecosystem Rule(s) and Goal(s) (101), which may be a limited or extensive set of rules and goals that can be used to set requirements or limitations on the GC that may be utilized in the GCMS or the results of the GE. The Ecosystem Rules and Goal(s) (101) may be stored in electronic memory, a rules/goals database, or the like, for example. Moreover, these Ecosystem Rules and Goals (101) may cover a wide variety of things including or excluding, increasing or decreasing, by way of example, but not limitation: carbon (or other elements/compounds) capture rates, carbon (or other elements/compounds) sequestration rates (amount and duration), carbon (or other elements/compounds) recalcitrant rates, resource requirements, resource consumption rates, process net carbon (or other elements/compounds) capture and sequestration rates (amount and duration), resulting GC albedo, ability to measure and report on carbon (or other elements/compounds) capture rates, GC inclusions, exclusions, placements, prioritization, weighting based on; GC source, GC type, primary/secondary GC, sexual/asexual reproduction, sterility, viability, intellectual property restrictions or requirements, licenses, date of creation, geographic source, maximum GC set length, minimum GC set length, maximum number of GC samples, minimum number of GC samples, quality of GC, business rules/goals, legal requirements, ethical considerations, individualized or grouped preferences, and the like, either individually or as a set and these can each be prioritized. While discussed further below, there may be different GC modification rule or goal sets based on elements, including but not limited to; source of the GC, impact of the GC, ethics, legal considerations, business considerations, impacts on future generations, impact on the broader ecosystem, and the like. The different variable characteristics that drive the selection of the GC may be weighted in any proportion as deemed appropriate such that GC items may be specifically included, excluded, prioritized, or given a likelihood of being included in a final modified GC. Cumulatively, the Ecosystem Rules and Goal(s) (101) provide the definition of what GC is qualified to be included in the Base Genetic Structure (108) and even considered to be included in the Finalized Genetic Structure (111). Furthermore, Ecosystem Rules and Goal(s) (101) is where the goal(s) and rule(s) of the process are set and these rules(s) and goal(s) (and the relative achievement of these goal(s)) influence how the rest of the process works.

The Ecosystem Rules and Goal(s) (101) provides the first set of information that helps to create the Base Genetic Structure Rules (102). The second set of information that also helps to create the Base Genetic Structure Rules (102) is Genetic Structure Results (103). The Genetic Structure Results (103) is an interpretation of the results of the GCMS (100) system. This review may be done by computer(s), by human(s), or a combination of both (e.g., user(s)). Please note that for an entirely new genetic modification process there may be no Genetic Structure Results, or there may be results from other related genetic modifications. Alternatively, if the example genetic modification is an iterative process there may be information related to the earlier cycles or other related modification processes. This information may be dynamically altered in real time (or near real time), continuously, or periodically as additional information is collected especially as additional iterations of the same or similar genetic modification(s) processes are reviewed. This information can cover a wide variety of topics including but not limited to the performance of genetic modification relative to the broader goal(s) and rule(s). These are often dealing with exclusions or inclusions of various genetic structures (and the manifestations of them). The combination of the Ecosystem Rules and Goal(s) (101) and the Genetic Structure Results (103) form the Base Genetic Structure Rules (102). This set of Base Genetic Structure Rules (102) provides the starting point for a structure of GC that satisfies all the broader rules and goals as well as the results from earlier cycles of the process (if any).

The Genetic Structure Results (103) provides the first set of information that helps to create the Predictive Rule (104) sets. The second set of information that also helps to create the Predictive Rules (104) is Other Historical Results (105). As discussed earlier, as results of the performance of the given genetic modification process(es) GE (if any) are created they are assembled continuously or periodically (including in real time or near real time) and collected in the Genetic Structure Results (103). Similarly results of the performance of any other genetic modification processes GE (if any) are created they are assembled continuously or periodically (including in real time or near real time) and collected in the Other Historical Results (105). It should be noted that these results sets can be immediate results, short term results, long-term results (individually or in combination) and their relationship to the goal(s), or results of GE activities outside of this given process (but still have data that is of interest to this process). This result information helps to take into consideration the impact of complex interdependent systems and otherwise unanticipated circumstances on the results of genetic modifications. The Predictive Rules (104) may combine these two data sets that provide a broad collection of information about the results of various genetic structures. This collection of information and calculations by the Predictive Rules (104) may function as a decision engine and can provide the basis for creating high quality predictions of the results of various genetic structures and help to identify sets of structures/GE that are most likely to provide the desired results.

The Base Genetic Structure Rules (102) and the Predictive Rules (104) combine to create the Genetic Structure Rule Set (106). This set combines the goal(s)/rule(s) of the process and predictive information to calculate the genetic structures/GE that most likely provide results to achieve those goal(s) and adhere those rule(s). This is the theoretical “best guess” genetic structure or structures (GEs). It should be noted that due to the fact that a great deal of the genetic structure/GE of many organisms are not well understood the “best guess” may just be that—though through an iterative process with multiple cycles (in series or parallel) the quality of these genetic structures/GE continues to improve. Furthermore, these “best guesses” may be created, reviewed, or altered by one or more humans or computers, or any combination of these (e.g., user(s)).

To create the Base Genetic Structure (108) it requires the combination of one or more elements including the Existing Genetic Content (107), New Genetic Content (109), and Genetic Content to be Removed (110). It should be noted that the Existing Genetic Content (107), the Base Genetic Structure (108), the New Genetic Content (109), and Genetic Content to be Removed (110) may each be a physical repository (separate or combined) containing GC, or each may be a digital database (separate or combined) storing digital data of GC (or a combination of physical and digital). The Existing Genetic Content (107) may be the primary building blocks of genetic code (gene bases), RNA, DNA, full genomes, entire organisms, populations of organisms, or anything (or portion of a thing) in between. In general, this is the GC that started with to be edited. Based on the information from the Genetic Structure Rule Set (106) New Genetic Content (109) is identified that should be added (and the location of the addition(s) of new GC) to the Existing Genetic Content (107). This additional GC could be as small as a single base element edit (A, C, G, & T or their analogues, including but not limited to uracil in RNA), or as big as an entire genome substitution (or anything in between). Additionally, the additional GC may also be a secondary organism, which in turn may be genetically edited through a similar process. It should be noted that it may be the case that no GC is added as part of the GE, or it may be the case that GC is just structurally rearranged (e.g., moved or re-positioned). Similarly, but generally oppositely, the Genetic Content to be Removed (110) is the GC that should be removed to ensure that the modified GC meets the structure identified in the Genetic Structure Rule Set (106). It should further be noted that it may be the case the GE is exclusively the removal (or knockout) of specific parts of the GC. In the exemplary system and method specific parts of the GC may be removed to reduce the buoyancy of the seaweed (increasing the likelihood of it sinking) or reduce the possibility of the seaweed sexually/asexually reproducing. It may be the case that GC identified to be removed is replaced with new GC to be added, but that is not necessarily the case—the modifications—removals (if any) and additions (if any) or moves/re-positions (if any) may not be replacements. Additionally, as part of this process GE deals not just with the content of any edit but also the location of any edit. In general the Base Genetic Structure (108) acts as a GE engine that is configured to edit genetic content information to have it comply with or achieve given rules or goals (at the organism, population, species, or ecosystem level), as well as, edit genetic content to ensure that the edited genetic content is coordinated and congruent with the edited genetic content information, in order to comply with or achieve the rules or goals. Furthermore, there may be multiple edits being done in an individual modification process, there may be multiple edits occurring in parallel, and all edits may be performed on primary (host) GC or secondary GC.

After the completion of the final GE, the modified genetic material is allowed to progress to exhibit the results of the edit as part of the Final Structure (111). This may be any of the types of genetic material including but not limited to, just a piece of genetic material, an organism, multiple generations of an organism, a community of the organisms, or the broader ecosystem. The results of the GEs are reviewed as part of the Evaluation of Results (112). This evaluation process may also take into consideration the several types of experimental error (systematic or random) that may have occurred in the process. This system reviews the results and reports them back to the Genetic Structure Results (103). It should be noted again that the final genetic structure can exist just in the physical world, just in the digital world, or a combination of both. Furthermore, this process of genetic modification and evaluation of the results may be done by a human, a computer, another system, or a combination of these (e.g., user(s)). Additionally, the entire process may run once or several times in full or in part in order to arrive at results that achieve the goal(s) (or do not fully achieve the goal(s)).

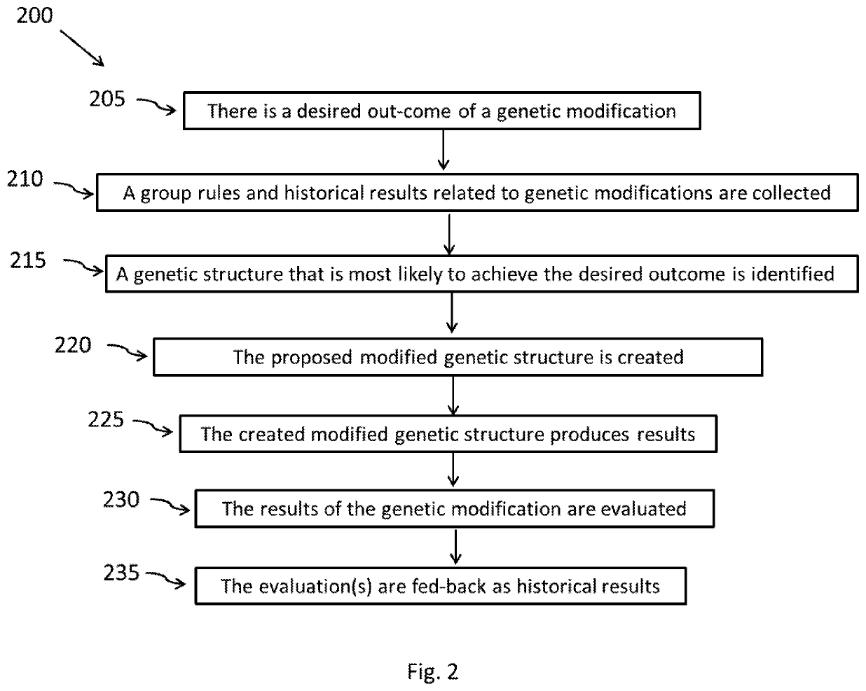

FIG. 2 illustrates a flowchart for a method according to an exemplary first embodiment. Initially, there is a desired outcome of a genetic GE to GC (205). There can be a wide variety of desired outcomes of genetic modification but there are challenges in achieving the desired outcomes due to the fact that the impact of GEs are often not fully understood, there are unanticipated conditions, unintended consequences, and the changes often occur as part of complex interrelated systems. The goal of this process is to take into consideration the unpredictable nature of GEs and produce genetic structure(s) that are more likely to achieve the goal(s) of the genetic modifications.

Once the desired result(s) of a genetic modification is identified a set of rules and historical results related to other genetic modifications are collected (210) to help educate and inform the decisions around the current genetic modification in order to facilitate the achievement of the goal(s) or desired results. As described earlier the rules and goal(s) can be broadly defined. The historical results (if any) may be results from earlier cycles of the same process, as well as historical results from other related genetic modifications (if any) may be used to help to identify a genetic structure that is most likely to achieve the desired outcome (215). This could also be referred to as a “best guess” genetic structure to achieve the goal(s) of the method. But due to the precise nature of GEing, if the “best guess” solution is not fully successful it may not just fail to achieve the goal(s) it may also be highly detrimental to the broader environment—a slight mistake in the GE may result in a big miss to the desired outcome. At this point a GE is applied to the GC to create the modified GC (220).

In the exemplary case the modified genetic structure(s) are created by manipulation of genetic material. This may be achieved by any of the GEing techniques, or by a combination of multiple editing techniques in series or parallel all at once or over time (even over generations of the genetic material or generations of the organism). This process of editing produces the results (225). These results are then evaluated in comparison to the original goal(s) (230). It should be noted that this editing may be a single specific set of edits done to one set of genetic material or a variety of sets of edits done to multiple sets of genetic material (and may include multiple organisms individually or collectively). Again, it should be noted that the edits may occur with actual biological material, digital data, or a combination of the two.

In the example case, these results may be considered successful (achieving the original desired outcomes), unsuccessful (not achieving the original desired outcomes), mixed (achieving of one or more of the original desired outcomes and not achieving one or more of the original desired outcomes, or having some unintended negative or positive result), or indeterminate (not clearly achieving or failing to achieve the original desired outcomes or other result). Please note that because the edits may be done on multiple sets, the results may be a mix of all of these. Additionally, this evaluation of the results may be done by a human, a computer, another system, or a combination of any of these (e.g., user(s)). These results are fed back (235) into the historical results (210) to help inform any subsequent cycles of the example method. This entire process may be repeated several times completely or in parts. Furthermore, all or some of the phases or complete cycles may occur in a physical, digital, or mixed manner. Please note, this method does not require any explicit user to initiate, complete, or recycle this process.

As an example, but not limitation, an exemplary embodiment of the present invention may follow the following progression: Process goals and ecosystem rules are identified in the example case—the goals may be the reducing environmental carbon dioxide by way of capturing the carbon dioxide in the structure of seaweed that grows (via photosynthesis) and identifying a genetic modification (GE) that causes it to lose buoyancy and sink to the bottom of deep water where the carbon is sequestered for a long period of time, and the ecosystem rule may be that the GE seaweed is not able to reproduce in such a way that it leads to significant negative impacts on the ecosystem. In light of these goals and rules current knowledge of historical tests in this field of study (if any), broader historical genetic information, understanding of GE causes and effects, and fundamental biologic genetic rules are combined to arrive at a “best guess” genetic structure that satisfies all of the limitations but has the best chance of achieving the goals. In this example case it may be a sargassum seaweed with specific edits that should be made to it (in a similar example case it may be kelp). The existing sargassum GC is the starting point and possibly it is thought that no additional GC needs to be added but rather some specific portion of the original GC is GEed to remove or knockout a specific portion of the GC to allow the sargassum to perform as desired. The “best guess” genetic structure may be arrived at by way of computer modeling of the genetic content, the genetic editing, and the results. This modeling may be done by way of traditional computing, quantum computing, or AI/ML, and may be the product of multiple iterative processes. This may result in genetic content information that may be the genetic content structure that the physical genetic content is edited to match with in a coordinated and congruent way. The physical GE occurs and resulting modified sargassum is grown and evaluated to see if the modified sargassum meets the goals while satisfying the rules. It is most likely that following the first process cycle the resulting modified sargassum does not perfectly meet the goals or rules and the process starts over again, but the knowledge from the first cycle helps to inform the second cycle and the process continues sequentially or in parallel until a good modified sargassum candidate is identified. Then the general process is to grow the modified in very small lab quantities, and if all goes well there to grow the sargassum in large lab tanks and if all continues to go well to then move onto open ocean test environments. All along the way continuous evaluation occurs looking for key performance metrics (e.g., carbon capture rate, other element capture rate, growth rate, nutrient requirements (including but not limited to iron, nitrogen, nitrates, phosphates, etc.), growth area to captured carbon mass, seaweed growth area albedo, ease monitoring & measurement, length of life cycle till sinking, various kinds of pneumatocyst failures (including but not limited to, overgrowth causing failure, loss of airtight seal, or ceasing adding gas to the pneumatocysts, etc.), decomposition resistance, rate of sinking, mass of carbon that sinks to the bottom, length of sequestration, rates of decay, sterility/ability to reproduce/replicate, ability to cross-pollinate, overall impact on the environment, unintended consequences, etc.). Furthermore, this same process may be applied with a combination of one or more organisms where the set of organisms works in a coordinated manner to achieve the goals consistent with the rules. For example, but not limitation, it may be found that the introduction of a modified coral to grow on the sargassum efficiently increases the density at such a rate that the sargassum/coral combination sinks in such a way that the mass of carbon sequestered per timer period per area is optimized while acting within the rules. Based on the evaluation of the performance of the GE GC a procedure of continuous improvement may be put in place following this system and method.

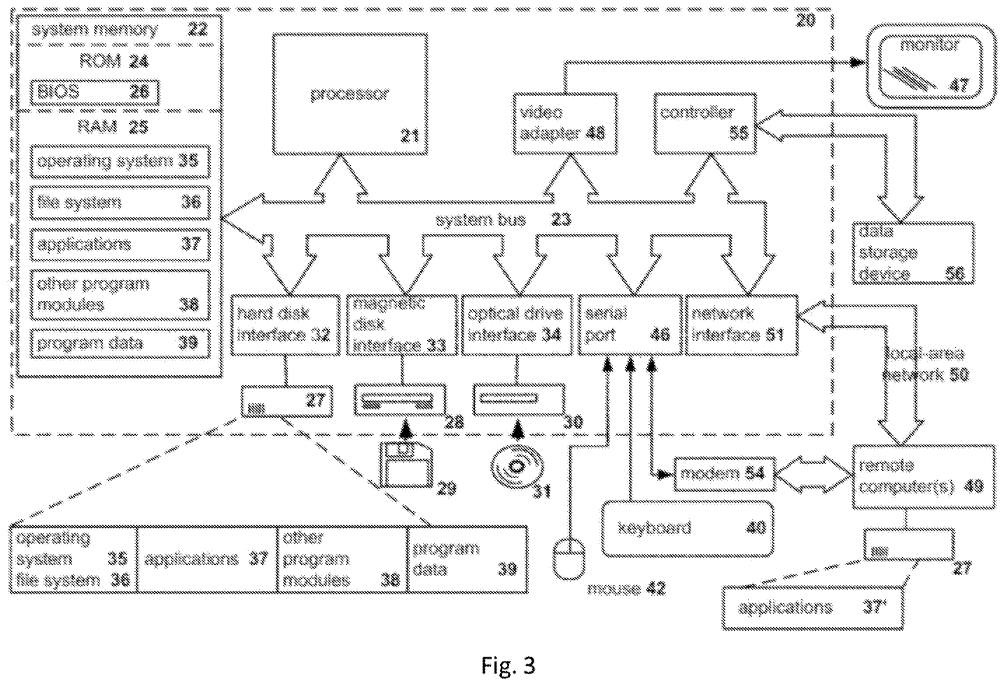

FIG. 3 illustrates an example of a general-purpose (classical or traditional) computer system (which may be a personal computer, a server, or a plurality of personal computers and servers) on which the disclosed system and method can be implemented according to an example aspect. It should be appreciated that the detailed general-purpose computer system can correspond to the GCMS (100) described above with respect to FIG. 1 to implement the algorithms described above. This general-purpose computer system (processor and storage) may exist in a single physical location, with a broadly distributed structure, virtually as a subset of larger computing systems (e.g., in the computing “cloud”), or a combination of any of these. Please note this is provided as an example not a limitation and the example embodiment may also use a quantum computing system in place of the general-purpose computer, or a quantum computer could be used in conjunction with the general-purpose computer. This combination may be performed in parallel, or series, or both, and similarly there may be multiple general-purpose computers or quantum computers used.

As shown, the computer system 20 includes a central processing unit 21, a system memory 22 and a system bus 23 connecting the various system components, including the memory associated with the central processing unit 21. The central processing unit 21 can be provided to execute software code (or modules) for the one or more set of rules discussed above which can be stored and updated on the system memory 22. Additionally, the central processing unit 21 may be capable of executing traditional computing logic, quantum computing, or a combination of both. Furthermore, the system bus 23 is realized like any bus structure known from the prior art, including in turn a bus memory or bus memory controller, a peripheral bus, and a local bus, which is able to interact with any other bus architecture. The system memory includes read only memory (ROM) 24 and random-access memory (RAM) 25. The basic input/output system (BIOS) 26 includes the basic procedures ensuring the transfer of information between elements of the personal computer 20, such as those at the time of loading the operating system with the use of the ROM 24.

As noted above, the rules described above can be implemented as modules according to an exemplary aspect. As used herein, the term “module” refers to a real-world device, component, or arrangement of components implemented using hardware, such as by an application specific integrated circuit (ASIC) or field-programmable gate array (FPGA), for example, or as a combination of hardware and software, such as by a microprocessor system and a set of instructions to implement the module's functionality, which (while being executed) transform the microprocessor system into a special-purpose device. A module can also be implemented as a combination of the two, with certain functions facilitated by hardware alone, and other functions facilitated by a combination of hardware and software. In certain implementations, at least a portion, and in some cases, all, of a module can be executed on the processor of a general-purpose computer. Accordingly, each module can be realized in a variety of suitable configurations and should not be limited to any example implementation exemplified herein.

The personal computer 20, in turn, includes a hard disk 27 for reading and writing of data, a magnetic disk drive 28 for reading and writing on removable magnetic disks 29 and an optical drive 30 for reading and writing on removable optical disks 31, such as CD-ROM, DVD-ROM and other optical information media. The hard disk 27, the magnetic disk drive 28, and the optical drive 30 are connected to the system bus 23 across the hard disk interface 32, the magnetic disk interface 33 and the optical drive interface 34, respectively. The drives and the corresponding computer information media are power-independent modules for storage of computer instructions, data structures, program modules and other data of the personal computer 20. Moreover, it is noted that any of the storage mechanisms (including data storage device 56, which may be amongst other things, physical hardware, CDN(s), or the “cloud”) can serve as the storage of the media Content, including the Available Content Library (111) described above, according to an exemplary aspect as would be appreciated to one skilled in the art.

The present disclosure provides the implementation of a system that uses a hard disk 27, a removable magnetic disk 29 and/or a removable optical disk 31, but it should be understood that it is possible to employ other types of computer information media 56 which are able to store data in a form readable by a computer (solid state drives, flash memory cards, digital disks, random-access memory (RAM) and so on, locally and/or remotely), which are connected to the system bus 23 via the controller 55.

The computer 20 has a file system 36, where the recorded operating system 35 is kept, and also additional program applications 37, other program modules 38 and program data 39. The user is able to enter commands and information into the personal computer 20 by using input devices (keyboard 40, mouse 42). Other input devices (not shown) can be used: microphone, joystick, game controller, scanner, other computer systems, and so on. Such input devices usually plug into the computer system 20 through a serial port 46, which in turn is connected to the system bus, but they can be connected in other ways, for example, with the aid of a parallel port, a game port, a universal serial bus (USB), a wired network connection, or wireless data transfer protocol. A monitor 47 or other type of display device is also connected to the system bus 23 across an interface, such as a video adapter 48. In addition to the monitor 47, the personal computer can be equipped with other peripheral output devices (not shown), such as loudspeakers, a printer, and so on.

The personal computer 20 is able to operate within a network environment, using a network connection to one or more remote computers 49, which can correspond to the remote viewing devices, i.e., the IP connected device (e.g., a smartphone, tablet, personal computer, laptop, media display device, or the like). Other devices can also be present in the computer network, such as routers, network stations, peer devices or other network nodes.

Network connections 50 can form a local-area computer network (LAN), such as a wired and/or wireless network, and a wide-area computer network (WAN). Such networks are used in corporate computer networks and internal company networks, and they generally have access to the Internet. In LAN or WAN networks, the personal computer 20 is connected to the network 50 across a network adapter or network interface 51. When networks are used, the personal computer 20 can employ a modem 54 or other modules for providing communications with a wide-area computer network such as the Internet or the cloud. The modem 54, which is an internal or external device, is connected to the system bus 23 by a serial port 46. It should be noted that the network connections are only examples and need not depict the exact configuration of the network, i.e., in reality there are other ways of establishing a connection of one computer to another by technical communication modules, such as Bluetooth.

In various aspects, the systems and methods described herein may be implemented in hardware, software, firmware, or any combination thereof. If implemented in software, the methods may be stored as one or more instructions or code on a non-transitory computer-readable medium. Computer-readable medium includes data storage. By way of example, and not limitation, such computer-readable medium can comprise RAM, ROM, EEPROM, CD-ROM, Flash memory or other types of electric, magnetic, or optical storage medium, or any other medium that can be used to carry or store desired program code in the form of instructions or data structures and that can be accessed by a processor of a general-purpose computer.

In the interest of clarity, not all of the routine features of the aspects are disclosed herein. It will be appreciated that in the development of any actual implementation of the present disclosure, numerous implementation-specific decisions must be made in order to achieve the developer's specific goal(s), and that these specific goal(s) will vary for different implementations and different developers. It will be appreciated that such a development effort might be complex and time-consuming but would nevertheless be a routine undertaking of engineering for those of ordinary skill in the art having the benefit of this disclosure.

It is noted that terms “compromises,” “comprising,” “includes,” “including,” “has,” “having” or any other variation thereof, are intended to cover a non-exclusive inclusion. For example, a process, system, method, article, or apparatus that comprises a list of elements is not necessarily limited to only those elements but may include other elements not expressly listed or inherent to such processes, systems methods, articles, or apparatus. Further, unless expressly stated to the contrary, “or” refers to an inclusive or and not to an exclusive or. For example, a condition A or B is satisfied by any one of the following: A is true (or present), and B is false (or not present), A is false (or not present), and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Furthermore, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Upon reading this disclosure, those of skill in the art will appreciate still additional alternative systematic and functional designs. Thus, while particular embodiments and applications have been illustrated and described herein, it is to be understood that the disclosed embodiments are not limited to the precise construction and components disclosed herein. Various modifications, changes, and variations, which will be apparent to those skilled in the art, may be made in the arrangement, operation, and details of the system and method disclosed herein without departing from the spirit and scope defined in the claims.

Furthermore, it is to be understood that the phraseology or terminology used herein is for the purpose of description and not of restriction, such that the terminology or phraseology of the present specification is to be interpreted by those skilled in the art in light of the teachings and guidance presented herein, in combination with the knowledge of the skilled in the relevant art(s). Moreover, it is not intended for any term in the specification or claims to be ascribed an uncommon or special meaning unless explicitly set forth as such.

The various aspects disclosed herein encompass present and future known equivalents to the known modules referred to herein by way of illustration. Moreover, while aspects and applications have been shown and described, it would be apparent to those skilled in the art having the benefit of this disclosure that many more modifications than mentioned above are possible without departing from the inventive concepts disclosed herein.

1. A system for selecting and editing at least a part of existing genetic content of at least one algae that captures carbon via photosynthesis in order to achieve at least one goal of causing the genetically edited algae to lose buoyancy due to pneumatocyst failure in at least one point in its life cycle, the system comprising:
at least one electronic database configured to store information related to at least one set of existing algae genetic content configured to be genetically edited by way of at least one of the addition, removal, or re-positioning of at least one set of new or existing algae genetic information related to at least one set of existing algae genetic content stored in at least one electronic database;
at least one processor with software instructions stored therein configured such that, when executed by the at least one processor, configure the at least one processor to execute:
a base genetic structure rules engine configured to generate at least one set of edited algae genetic information structure based on at least one goal;
a base algae genetic structure engine configured to generate at least one set of genetically edited algae genetic content by modifying existing algae genetic content by at least one of;
adding at least one set of new genetic content;
removing at least one set of existing genetic content;
re-positioning at least one set of existing genetic content;
such that the at least one set of edited algae genetic content is congruent with the at least one set of edited algae genetic information structure;
wherein the at least one set of genetically edited algae genetic content is in compliance with the at least one goal.

2. The system according to claim 1, wherein the at least one algae to be genetically modified is sargassum.

3. The system according to claim 1, wherein at least one genetic modification is related to restricting the ability of the at least one algae to reproduce.

4. The system according to claim 1, wherein the at least one genetic modification is related to increasing the ability of the at least one algae to sequester carbon.

5. The system according to claim 1, wherein at least one genetic modification is done to at least one secondary organism.

6. The system according to claim 1, wherein the entire genetic modification is performed in a digital environment.

7. A method for selecting and editing at least a part of existing genetic content of at least one algae that captures carbon via photosynthesis in order to achieve at least one desired outcome of causing the genetically edited algae to lose buoyancy due to pneumatocyst failure in at least one point in its lifecycle, the method comprising:
storing in at least one electronic database configured to store at least one set of genetic information related to existing genetic content configured to be genetically edited by way of at least one of; the addition, removal, or re-positioning of one set of new or existing genetic information related to existing algae genetic content stored in at least one electronic database;
generating by at least one processor with software instructions stored therein that, when executed by the at least one processor, configure the at least one processor to execute:
generating by at least one base genetic structure rules engine configured to generate at least one set of edited algae genetic information structure based on at least one desired outcome;
applying at least one base genetic structure rules engine configured to generate at least one set of genetically edited algae genetic content by at least one of;
adding at least one set of new genetic content;
removing at least one set of existing genetic content;
re-positioning at least one set of existing genetic content;
such that the at least one set of edited algae genetic content that is congruent with the at least one set of edited algae genetic information structure;
wherein the at least one set of genetically edited algae genetic content is in compliance with the at least one desired outcome.

8. The method according to claim 7, wherein the at least one algae to be genetically modified is sargassum.

9. The method according to claim 7, wherein at least one genetic modification is related to restricting the ability of the at least one algae to reproduce.

10. The method according to claim 7, wherein the at least one genetic modification is related to increasing the ability of the at least one algae to sequester carbon.

11. The method according to claim 7, wherein at least one genetic modification is done to at least one secondary organism.

12. The method according to claim 7, wherein the entire genetic modification is performed in a digital environment.
